(19) United States
(12) Patent Application Publication
(10) Pub. No.: US 2023/0277257 A1
(43) Pub. Date: Sep. 7, 2023

(54) ROBOTIC IMAGING SYSTEM WITH ORBITAL SCANNING MODE

(71) Applicant: Alcon Inc., Fribourg (CH)

(72) Inventors: Patrick Terry, Goleta, CA (US); Ashok Burton Tripathi, Santa Barbara, CA (US)

(21) Appl. No.: 18/175,013

(22) Filed: Feb. 27, 2023

Related U.S. Application Data
(60) Provisional application No. 63/315,870, filed on Mar. 2, 2022.

(51) Int. Cl. A61B 34/30; A61B 90/00; H04N 23/67

(52) U.S. Cl. CPC A61B 34/30; A61B 90/361; A61B 90/37; H04N 23/675; A61B 2090/371

(57) ABSTRACT

A robotic imaging system includes a stereoscopic camera configured to record left and right images of a target site. A robotic arm is operatively connected to the stereoscopic camera, the robotic arm being adapted to selectively move the stereoscopic camera relative to the target. The stereoscopic camera includes an optical assembly having at least one lens and defining a working span. The optical assembly has at least one focus motor adapted to move the at least one lens to selectively vary the working span. The robotic imaging system includes a controller having a processor and tangible, non-transitory memory on which instructions are recorded. The controller is adapted to selectively execute an orbital scanning mode causing the robotic arm to sweep an orbital trajectory at least partially circumferentially around the eye while maintaining focus.

### INTRODUCTION

The present disclosure relates generally to a robotic imaging system. More specifically, the disclosure relates to an orbital scanning mode in a robotic imaging system. Various imaging modalities are commonly employed to image different parts of the human body, including stereoscopic microscopes. Some surgical procedures may require movement of the camera over multiple regions in real-time and the acquisition of focused images. Traditional microscopes do not have the capability of performing this motion. Additionally, it is challenging to provide minimally invasive imaging techniques for scanning over delicate and small regions of the human body.

### SUMMARY

Disclosed herein is a robotic imaging system for imaging a target site. The robotic imaging system includes a stereoscopic camera configured to record left and right images of the target site for producing at least one stereoscopic image of the target site. A robotic arm is operatively connected to the stereoscopic camera, the robotic arm being adapted to selectively move the stereoscopic camera relative to the target. The stereoscopic camera includes an optical assembly having at least one lens and defining a working span. The optical assembly has at least one focus motor adapted to move the at least one lens to selectively vary the working span. A controller is in communication with the stereoscopic camera and has a processor and tangible, non-transitory memory on which instructions are recorded.

The controller is adapted to selectively execute an orbital scanning mode causing the robotic arm to sweep an orbital trajectory at least partially circumferentially around the eye while maintaining focus. The controller may be configured to determine a change in target depth from an initial target position, the change in the target depth being defined as a displacement in position of the target site along an axial direction. The controller is configured to update a specific focal length based in part on the change in the target depth. The target site may include an orra serrata of the eye.

The orbital trajectory may be defined in a spherical coordinate axis defining a first spherical angle and a second spherical angle. The controller is adapted to change a view angle of the orbital trajectory by keeping the first spherical angle constant while iterating the second spherical angle until a desired viewing angle is reached. The controller is adapted to selectively command the orbital trajectory by iterating the first spherical angle between a predefined starting angle and a predefined ending angle while keeping the second spherical angle constant at the desired viewing angle. In some embodiments, the orbital trajectory at least partially forms a circle. In some embodiments, the orbital trajectory at least partially forms an ellipsoid. The orbital trajectory may subtend an angle between about 180 degrees and 300 degrees. The orbital trajectory may subtend an angle of about 360 degrees.

The controller may be configured to center the stereoscopic camera on a reference plane of the eye and estimate a first working span to a reference surface of the eye. The controller may be adapted change a view vector of the stereoscopic camera to a desired viewing angle. The controller may be configured to lock a respective position of each target point along the orbital trajectory by restricting the respective position of the stereoscopic camera to an outer surface of a virtual sphere, the virtual sphere defining a radius equal to the specific focal length. The specific focal length may be based in part on a desired viewing angle, a dimension of the eye and a first working span.

The controller may be configured to determine a change in height of the stereoscopic camera from an initial camera position, the change in the height being defined as a displacement in position of the stereoscopic camera along an axial direction. The controller may be configured to update the specific focal length based in part on the change in the height of the stereoscopic camera.

When the robotic arm is no longer moving, the controller may be configured to determine motor commands for the at least one focus motor corresponding to a maximum sharpness position. The maximum sharpness position is based on one or more sharpness parameters, including a sharpness signal, a maximum sharpness signal and a derivative over time of the maximum sharpness. In each update cycle, the controller may be configured to inject respective delta values to respective coordinate positions of the orbital trajectory.

Disclosed herein is a stereoscopic imaging system for imaging a target site in an eye. The stereoscopic imaging system includes a stereoscopic camera configured to record a left image and a right image of the target site for producing at least one stereoscopic image of the target site. A robotic arm is operatively connected to the stereoscopic camera, the robotic arm being adapted to selectively move the stereoscopic camera relative to the target site. The stereoscopic camera includes an optical assembly having at least one lens and defining a working span, the optical assembly having at least one focus motor adapted to move the at least one lens to selectively vary the working span. A controller is in communication with the robotic arm and has a processor and tangible, non-transitory memory on which instructions are recorded. The controller is adapted to selectively execute an orbital scanning mode causing the robotic arm to sweep an orbital trajectory at least partially circumferentially around the eye while maintaining focus. The target site may include an orra serrata of the eye.

The orbital trajectory may be defined in a spherical coordinate axis defining a first spherical angle and a second spherical angle. The controller is adapted to change a view angle of the orbital trajectory by keeping the first spherical angle constant while iterating the second spherical angle until a desired viewing angle is reached. The controller is adapted to selectively command the orbital trajectory by iterating the first spherical angle between a predefined starting angle and a predefined ending angle while keeping the second spherical angle constant at the desired viewing angle. The sharpness signal may be defined as a contrast between respective edges of an object in the at least one stereoscopic image. The maximum sharpness signal may be defined as the largest sharpness value observed during a scan period.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic fragmentary diagram of a robotic imaging system having a stereoscopic camera and a controller with an orbital scanning mode;

FIG. 2 is a schematic fragmentary diagram of example optical components of the stereoscopic camera of FIG. 1;

FIG. 3 is a schematic fragmentary sectional diagram of an eye;

FIG. 4 is a schematic fragmentary sectional diagram of the eye, through axis 4-4 of FIG. 3;

FIG. 5 is a schematic diagram of a virtual sphere employable by the robotic imaging system of FIG. 1; and

FIG. 6 is a flowchart of an example method for operating the orbital scanning mode of FIG. 1.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

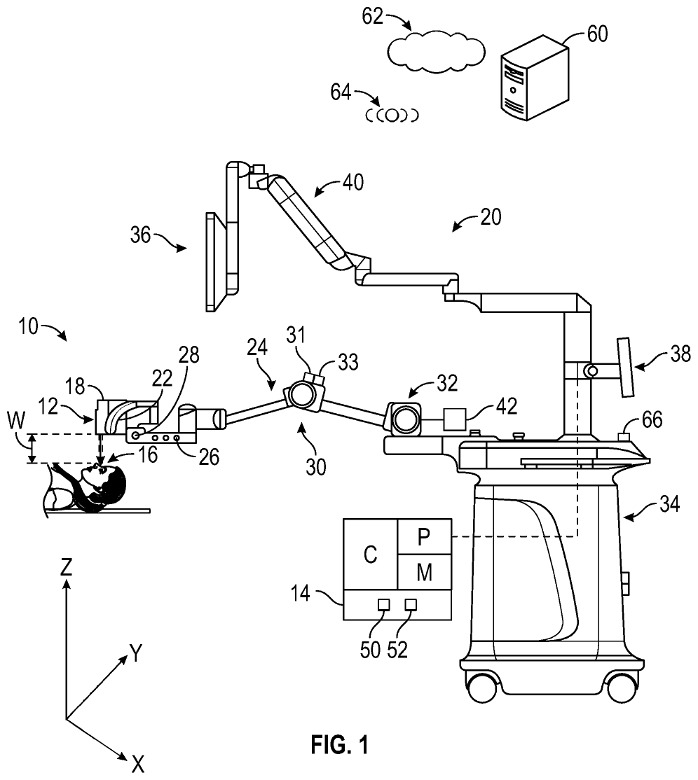

Referring to the drawings, wherein like reference numbers refer to like components, FIG. 1 schematically illustrates a robotic imaging system 10 having a stereoscopic camera 12 with an orbital scanning mode 14. The robotic imaging system 10 is configured to image a target site 16. The orbital scanning mode 14 allows a surgeon to view parts of the eye without actually touching the eye, thereby avoiding contact procedures such as scleral depression. This provides a quicker way to inspect the eye, decreasing case time and reducing potential trauma.

Referring to FIG. 1, the stereoscopic camera 12 is at least partially located in a head unit 18 of a housing assembly 20, with the head unit 18 configured to be at least partially directed towards the target site 16. The stereoscopic camera 12 is configured to record first and second images of the target site 16, which may be employed to generate a live two-dimensional stereoscopic view of the target site 16. The target site 16 may be an anatomical location on a patient, a laboratory biological sample, calibration slides/templates, etc.

Referring to FIG. 1, at least one selector 22 may be mounted on the head unit 18 for selecting specific features of the stereoscopic camera 12, such as magnification, focus and other features. The selector 22 may be employed to enable an operator to manually position the head unit 18. The robotic imaging system 10 may include a robotic arm 24 operatively connected to and configured to selectively move the head unit 18. Referring to FIG. 1, the head unit 18 may be mechanically coupled to the robotic arm 24 via a coupling plate 26. The operator may position and orient the stereoscopic camera 12 with assistance from the robotic arm 24. A sensor 28 may be operatively connected to the robotic arm 24 and/or coupling plate 26. The sensor 28 is configured to detect forces and/or torque imparted by an operator for moving the stereoscopic camera 12. The controller C can be adapted to calculate trajectories to move the robotic arm 24 spherically while maintaining a constant target viewpoint.

The robotic arm 24 may include one or more joints, such as first joint 30 and second joint 32, configured to provide further degrees of positioning and/or orientation of the head unit 18. The data from the sensor 28 may be employed to determine which joints of the robotic arm 24 should be rotated and how quickly the joints should be rotated, in order to provide assisted movement of the stereoscopic camera 12 that corresponds to the forces/torques provided by the operator. Referring to FIG. 1, a respective joint motor (such as joint motor 31) and a respective joint sensor (such as joint sensor 33), may be coupled to each joint. The joint motor 31 is configured to rotate the first joint 30 around an axis, while the joint sensor 33 is configured to transmit the position (in 3D space) of the first joint 30.

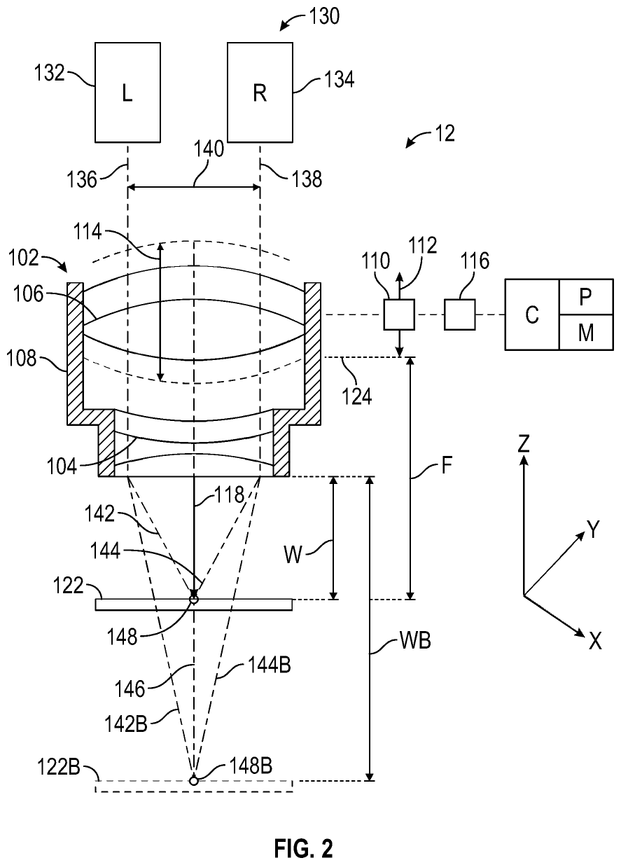

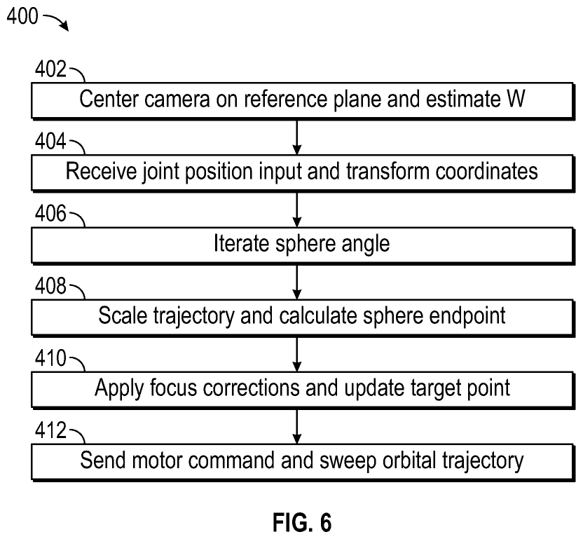

Referring to FIG. 1, the robotic imaging system 10 includes a controller C having at least one processor P and at least one memory M (or non-transitory, tangible computer readable storage medium) on which are recorded instructions for executing one or more sub-routines or methods, including a method 400 (described with respect to FIG. 6) of operating the orbital scanning mode 14. The memory M can store controller-executable instruction sets, and the processor P can execute the controller-executable instruction sets stored in the memory M. The method 400 provides the workflow, robotic motion, and focus motor adjustments to move the stereoscopic camera 12 while keeping the image in focus at all times, without having to physically move the eye 200 (see FIG. 2). Traditional microscopes do not have the capability of performing this motion.

Referring to FIG. 1, the robotic arm 24 may be controlled via the controller C and/or an integrated processor, such as a robotic arm controller 42. The robotic arm 24 may be selectively operable to extend a viewing range of the stereoscopic camera 12 along an X-axis, a Y-axis and a Z-axis. The head unit 18 may be connected to a cart 34 having at least one display medium (which may be a monitor, terminal or other form of two-dimensional visualization), such as first and second displays 36 and 38 shown in FIG. 1. Referring to FIG. 1, the controller C may be configured to process signals for broadcasting on the first and second displays 36 and 38. The housing assembly 20 may be self-contained and movable between various locations. The image stream from the stereoscopic camera 12 may be sent to the controller C and/or a camera processor (not shown), which may be configured to prepare the image stream for viewing. For example, the controller C may combine or interleave first and second video signals from the stereoscopic camera 12 to create a stereoscopic signal. The controller C may be configured to store video and/or stereoscopic video signals into a video file and stored to memory M. The first and second displays 36 and 38 may incorporate a stereoscopic display system, with a two-dimensional display having separate images for the left and right eye respectively. To view the stereoscopic display, a user may wear special glasses that work in conjunction with the first and second displays 36, 38 to show the left view to the user's left eye and the right view to the user's right eye.

Referring to FIG. 1, the first display 36 may be connected to the cart 34 via a flexible mechanical arm 40 with one or more joints to enable flexible positioning. The flexible mechanical arm 40 may be configured to be sufficiently long to extend over a patient during surgery to provide relatively close viewing for a surgeon. The first and second displays 36, 38 may include any type of display, such as a high-definition television, an ultra-high-definition television, smart-eyewear, projectors, one or more computer screens, laptop computers, tablet computers, and/or smartphones and may include a touchscreen.

The stereoscopic camera 12 is configured to acquire stereoscopic images of the target site 16, which may be presented in different forms, including but not limited to, captured still images, real-time images and/or digital video signals. “Real-time” as used herein generally refers to the updating of information at the same rate as data is received. More specifically, “real-time” means that the image data is acquired, processed, and transmitted at a high enough data rate and a low enough delay that when the data is displayed, objects move smoothly without user-noticeable judder or latency. Typically, this occurs when new images are acquired, processed, and transmitted at a rate of at least about 30 frames per second (fps) and displayed at about 60 fps and when the combined processing of the video signal has no more than about 1/30^{th} second of delay.

### I. Optical Components

Referring now to FIG. 2, an example layout of optical components of the stereoscopic camera 12 is presented. It is to be understood that other optical components or devices available to those skilled in the art may be employed. Images from the target site 16 are received at the stereoscopic camera 12 via an optical assembly 102, shown in FIG. 2. The optical assembly 102 includes a front lens 104 and a rear lens 106, within a housing 108.

Referring to FIG. 2, a focal plane 122 is located at a distance equal to a specific focal length F from a principal plane 124 of the optical assembly 102. Visualization of an object with the stereoscopic camera 12 above or below the focal plane 122 diminishes a focus of the object. It may be difficult to gauge the location of the principal plane 124, so a distance from the bottom surface of the housing 108 to the focal plane 122 can be designated as a working span W. The working span W accurately sets a plane of the target site 16 or scene that is in focus.

The optical assembly 102 is configured to provide a variable working span W (see FIG. 1) for the stereoscopic camera 12. Referring to FIG. 2, the controller C is adapted to selectively command a focus motor 110 to change the spacing between the rear lens 106 and the front lens 104. The focus motor 110 is movable (for example, along direction 112) to vary the working span W of the optical assembly 102. As noted above, the working span W may be referred to as the distance from the stereoscopic camera 12 to a reference plane where the target site 16 is in focus. In some embodiments, the working span W is the distance from the optical origin point of the stereoscopic camera 12 to a predefined reference plane in the target site 16. In some embodiments, the working span W is adjustable from 200 to 450 mm by moving the rear lens 106 via the focus motor 110.

Movement of the rear lens 106 relative to the front lens 104 also changes the specific focal length F, which is bounded by the maximum and minimum working span permitted by the hardware. A focal length can be defined as the distance between the rear lens 106 and the front lens 104 plus one-half the thickness of the front lens 104. In one example, the focus motor 110 is an electric motor. However, the focus motor 110 may be any type of linear actuator, such as a stepper motor, a shape memory alloy actuator or other type of actuator available to those skilled in the art.

In the embodiment shown, the rear lens 106 is movable along a direction 114 (Z-axis here) while the front lens 104 is stationary. However, it is understood that the front lens 104 may be movable or both the front lens 104 and rear lens 106 may be movable. The focus motor 110 may be selectively operable through the controller C and/or a motor controller 116. The stereoscopic camera 12 may include additional focus motors to independently move the front and rear lenses. The orientation of the stereoscopic camera 12 is indicated by a view vector 118 in 3D space.

In some embodiments, the front lens 104 is composed of a plano-convex lens and/or a meniscus lens. The rear lens 106 may comprise an achromatic lens. In examples where the optical assembly 102 includes an achromatic refractive assembly, the front lens 104 may include a hemispherical lens and/or a meniscus lens. The rear lens 106 may include an achromatic doublet lens, an achromatic doublet group of lenses, and/or an achromatic triplet lens. The optical assembly 102 may include other types of refractive or reflective assemblies and components available to those skilled in the art. The magnification of the optical assembly 102 may vary based on the working span W. For example, the optical assembly 102 may have a magnification of 8.9× for a 200 mm working span and a magnification of 8.75× for a 450 mm working span.

Referring to FIG. 2, imaging an object at the focal plane 122 develops a conjugate image located at infinity from a back or rear of the optical assembly 102. The optical assembly 102 is configured to provide left and right views of the target site 16, via an optical device 130. In some embodiments, the optical device 130 includes left and right optical units 132, 134 having respective sensors and optical devices. As shown in FIG. 2, the left and right optical units 132, 134 respectively generate a left optical path 136 and a right optical path 138, which are two parallel optical paths within the housing 108. The left and right optical units 132, 134 are transversely separated by a distance 140. In some embodiments, the distance 140 is between about 58 to 70 mm. External to the housing 108, the left optical path 136 and the right optical path 138 extend into a left optical axis 142 and a right optical axis 144, respectively, in slightly different directions from an optical axis 146 of the optical assembly 102. The left optical axis 142 and the right optical axis 144 coincide at the center of the field of view, at a target point 148. The target point 148 may be referred to as the “tip” of the stereoscopic camera 12 at the focal plane 122.

Adjusting the relative positions of the front lens 104 and rear lens 106 creates a new working span WB, that is located at the position of a new focal plane 122B. Referring to FIG. 2, the movement of the rear lens 106 causes a realignment of the left optical axis 142B and the right optical axis 144B, resulting in a relocated tip 148B of the stereoscopic camera 12.

Together, the front lens 104 and the rear lens 106 are configured to provide an infinite conjugate image for providing an optimal focus for downstream optical image sensors. In other words, an object located exactly at the focal plane of the target site 16 will have its image projected at a distance of infinity, thereby being infinity-coupled at a provided working span. Generally, the object appears in focus for a certain distance along the optical path from the focal plane. However, past the certain threshold distance, the object begins to appear fuzzy or out of focus.

The optical assembly 102 shown in FIG. 2 provides an image of the target site 16 for both the left and right optical paths 136, 138. In alternative embodiments, the optical assembly 102 may include a separate left and a separate right front lens 104 and separate left and a separate right rear lens 106. Further, each of the rear lenses 106 may be independently adjustable.

### II. Orbital Scanning Mode

Referring to FIG. 1, the controller C is adapted to selectively execute the orbital scanning mode 14 for the stereoscopic camera 12, which may be selectively initiated by a user. The orbital scanning modes 14 leverages the two available images (left and right) of the stereoscopic camera 12 to perform an orbital trajectory and maintain the focus of the image, without requiring laborious manual focusing.

In some embodiments, the orbital scanning mode 14 begins when an operator selects orbital scanning mode 14 (e.g., via an input device 66 such as a mouse, see FIG. 1), which causes an instruction message or signal to be transmitted to the controller C and/or the robotic arm controller 42. When an instruction is received, the example controller C and/or the robotic arm controller 42 may record the current working span, magnification, focus, and/or other optical parameters of the stereoscopic camera 12. The current position of the target site 16 may be determined from position data of the various joints of the robotic arm 24. The controller C and/or the robotic arm controller 42 may also record a current image of the target site 16.

The orbital scanning mode 14 may be used on any stereoscopic visualization device with a working span W that is variable, e.g., that is changeable. FIG. 1 shows a working span W, which may be defined as the distance between a reference plane and the focal plane of the target site 16. The working span W accordingly sets a plane of the target site 16 or scene that is in focus. In the example shown in FIG. 2, the reference plane is an outer surface of a front lens (not shown) in the optical assembly 102. The working span W may correspond to an angular field-of-view, where a longer working span results in a wider field-of-view or larger viewable area. Based in part on robotic arm input and dynamic image feedback, the orbital scanning mode 14 automatically adjust the working span W to achieve improved image quality in a variety of situations.

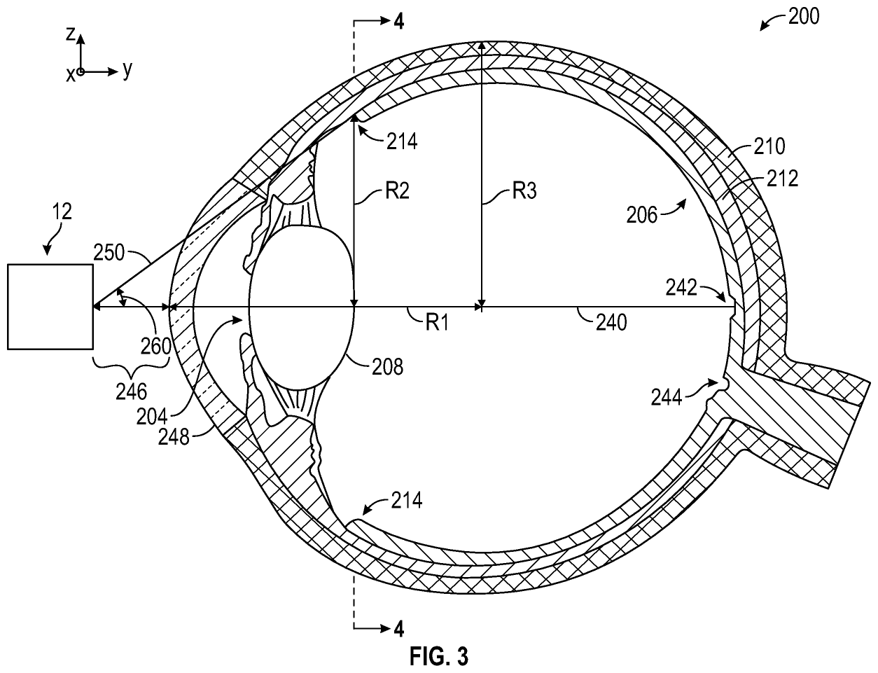

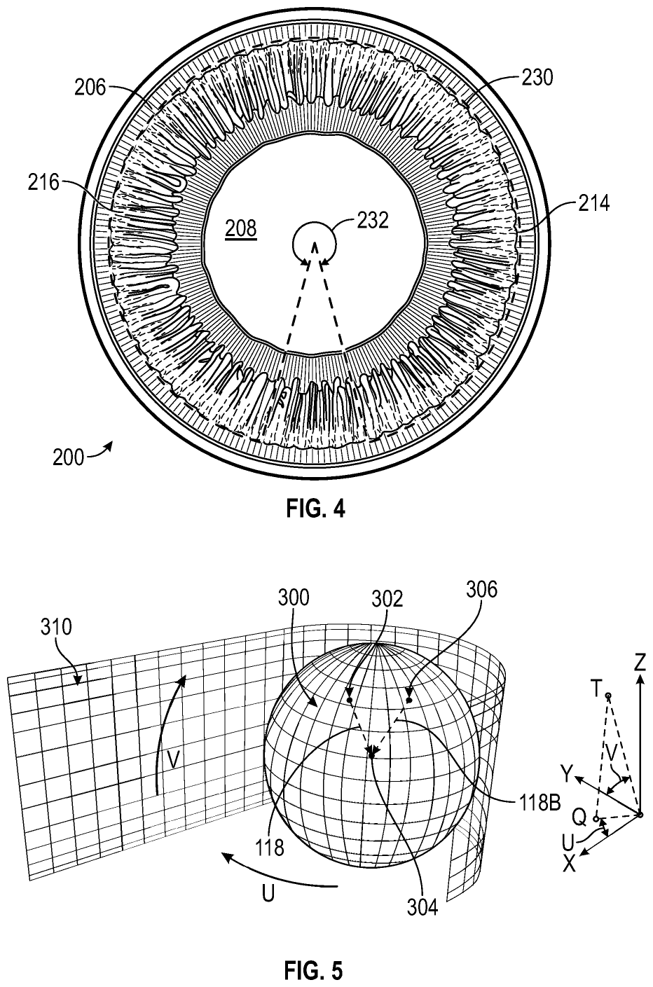

FIG. 3 is a schematic fragmentary sectional diagram of an eye 200 having a pupil 204 and retina 206. FIG. 4 is a schematic fragmentary sectional diagram through axis 4-4 of FIG. 3. Referring to FIGS. 3-4, the eye 200 includes a lens 208, sclera 210 and choroid 212. The sensory region of the eye 200 begins at the orra serrata 214. In the embodiment shown in FIGS. 3-4, the target site 16 (see FIG. 1) includes or covers the orra serrata 214 of the eye 200. However, it is understood that other portions of the eye 200 or human body may be imaged. The orra serrata 214 is a serrated junction between the retina 206 and the corona ciliaris 216 (see FIG. 4, also referred to as ciliary crown), defining a transition point between the non-sensory region and the multi-layered sensory region of the eye 200.

The robotic imaging system 10 provides surgeons with a way to perform an orbital scan of the eye 200, for example, starting at and/or viewing the orra serrata 214 (see FIGS. 3-4). This allows surgeons to quickly look for anomalies such as retina tears, breaks at periphery, residual vitreous, etc. The controller is programmed to move the stereoscopic camera 12 while keeping the image in focus at all times, without having to physically move the eye 200.

As described below, the operator/surgeon can center the robotic imaging system 10 on the eye 200, engage the orbital scanning mode 14, and the robotic imaging system 10 can move to view the orra serrata 214 of the eye 200. Once at the orra serrata 214, the surgeon can begin moving the robotic arm 24 in an orbital trajectory 230 that performs a scan circumferentially around the eye 200. Multiple orbital trajectories 230 may be performed.

The controller C may be adapted to provide an application programming interface (API) for starting and stopping each orbital trajectory of the orbital scanning mode 14. The shape of the orbital trajectory 230 may be modified based on the application at hand. In some embodiments, the orbital trajectory 230 at least partially forms a circle. In other embodiments, the orbital trajectory 230 at least partially forms an ellipsoid. Referring to FIG. 4, the orbital trajectory 230 may subtend an angle 232 between about 180 degrees and 300 degrees. In some embodiments, the angle 232 is about 350 degrees. The orbital trajectory 230 may include multiple 360-degree rotations, assuming the robotic arm 24 has sufficient joint limit clearance. Additionally, the orbital trajectory 230 may either stop short of a full rotation or traverse an irregular shape as it pulls the radius in to avoid hardware/joint limits.

The orbital scanning mode 14 enables the stereoscopic camera 12 to be moved around anywhere in 3D space (via the robotic arm 24), permitting changes in working span, while being locked and focused onto a specific point (e.g., target point 148 shown in FIG. 2) in the target site 16 in real-time. The orbital trajectory 230 may be defined in terms of a spherical coordinate system having a first spherical angle (U) and a second spherical angle (V), shown in FIG. 5 for an example location Tin XYZ space and its projection Q in the XY plane. FIG. 5 shows an example virtual sphere 300 employable by the controller C. The controller C and/or the robotic arm controller 42 of FIG. 1 enable an operator to move the stereoscopic camera 12 over the outer surface 302 of the virtual sphere 300 to an end location 306, while keeping the stereoscopic camera 12 pointed at the center 304 (as indicated by view vectors 118, 118B). The center 304 has the same coordinates in XYZ space as a selected point in the target site 16, such as target point 148 shown in FIG. 2.

Referring now to FIG. 6, a flowchart is shown of an example method 400 for implementing the orbital scanning mode 14 of FIG. 1. Method 400 may be embodied as computer-readable code or instructions stored on and partially executable by the controller C of FIG. 1. Method 400 need not be applied in the specific order recited herein and may be dynamically executed. Furthermore, it is to be understood that some steps may be eliminated.

Method 400 begins with block 402 of FIG. 5, where the stereoscopic camera 12 is centered on a reference plane 240 (see FIG. 3) of the eye 200, via the robotic arm 24 of FIG. 1. In some embodiments, referring to FIG. 3, the reference plane 240 extends through the fovea centralis 242. In other embodiments, the reference plane 240 can extend through the optic disc 244. Also, per block 402 of FIG. 6, the controller C is programmed to estimate a first working span 246 (W) to a surface 248 of the eye 200, as shown in FIG. 3. The controller C is programmed to increase the radius or specific focal length F (see FIG. 2) to a second working span 250 (see FIG. 3), which can be obtained as a sum of the first working span 246 and a dimension of the eye, e.g., first dimension R1 which may be the radius (anterior to posterior direction) of the eye 200 obtained from an eye model and/or anatomical data for the patient in question.

Proceeding from block 402 to block 404 of FIG. 6, the method 400 includes receiving input data, such as joint position coordinates of the robotic arm 24. In each update cycle, the controller C obtains as an input the current joint angles of the orbital trajectory 230, which may not be the same as the current joint angles of the robotic arm 24. Also, per block 404, the controller C is adapted to transform the joint position coordinates from the camera frame to a global frame, and into spherical coordinates defined by a first spherical angle (U) and a second spherical angle (V).

Advancing from block 404 to block 406 in FIG. 6, the method 400 includes iterating at least one of the first spherical angle (U) and the second spherical angle (V). First, the viewing angle of the orbital trajectory 230 can be changed by keeping the first spherical angle (U) fixed (e.g., at 0, 180 degrees or any other angle) and iterating the second spherical angle (V) from (V+ΔV) to (V−ΔV) until a desired viewing angle 260 is reached. The desired viewing angle 260 represents the angle required to view the orra serrata 214 and may be pre-programmed into the controller C based on an eye model and/or anatomical data. In some embodiments, changing the viewing angle can be part of a “hold-to-move” input knob/device that the surgeon can adjust to enable orbiting at a different view angle. The position of the target point 148 at the desired viewing angle 260 (V0) is locked via the controller C.

Second, orbits of the orbital trajectory 230 can be performed by holding the second sphere angle (V) constant at the desired viewing angle 260, while iterating movement along the first sphere angle (U) of the virtual sphere 300. The beginning and end of the orbit can be defined by a predefined starting angle (Uᵢₙᵢₜᵢₐₗ) and a predefined ending angle (U_{final}). Referring to FIG. 1, the robotic imaging system 10 may include an optical coherence tomography (OCT) module configured to obtain visualization data of the target site 16. The visualization data may be employed to guide the orbital trajectory 230, e.g., via selection of the desired viewing angle 260. U.S. application Ser. No. 17/108,458 (filed on Dec. 1, 2020), the contents of which is hereby incorporated by reference in its entirety, describes a system with an integrated visualization camera and optical coherence tomography module.

Proceeding from block 406 to block 408 in FIG. 6, the controller C is configured to scale the speed of the orbital trajectory 230 and calculate an end sphere point (e.g., end location 306 in FIG. 5). As noted above, the orbital scanning mode 14 restricts the position of the stereoscopic camera 12 to the outer surface of a virtual sphere, for example, outer surface 302 of a virtual sphere 300 shown in FIG. 5, based on the current position of the target site 16. The iterative movement along the first sphere angle (U) enables the end location 306 to be determined for an orbit input. A given point on the outer surface 302 is given by an equation that is a function of the first spherical angle (U) and the second spherical angle (V). In other embodiments, the virtual sphere 300 may be represented by a different shape and the outer surface 302 may be represented by a planar surface 310. Based on the type of input received, the end location 306 (of the stereoscopic camera 12) on the outer surface 302 may be obtained in different ways. For example, input can be received via an input device 66 (e.g., a joystick or mouse) as shown in FIG. 1. The controller C and/or the robotic arm controller 42 may be adapted to convert ‘up’, ‘down’, ‘left’, and ‘right’ from camera coordinates to robotic base frame coordinates, which are provided as X and Y vectors. The X and Y vectors are used by the controller C and/or the robotic arm controller 42 for directly determining how the stereoscopic camera 12 is to move on the virtual sphere 300 to determine the end location 306.

Also, per block 408, the controller C is adapted to calculate an amount of rotation needed for the stereoscopic camera 12 to maintain the lock at the coordinates of the target point 148 after the stereoscopic camera 12 has been moved. In other words, the controller C is configured to determine how the stereoscopic camera 12 is to be orientated given its new position on the virtual sphere 300 such that the view vector 118B of the end location 306 is provided at the same XYZ coordinates at the center of the virtual sphere 300 (corresponding to the selected point). The controller C and/or the robotic arm controller 42 are adapted to determine the joint angles of the robotic arm 24 and/or the coupling plate 26 needed to achieve the desired orientation.

Advancing from block 408 to block 410 in FIG. 6, the controller C is configured to apply a number of corrections and update the coordinates of the target point 148. The location of the target point 148, having coordinates [Xtarget, Ytarget, Ztarget], in the orbital trajectory 230 may be obtained from the first spherical angle (U) and a second dimension R2 of the eye 200, as follows: [Xtarget=R2*Cosine(U), Ytarget=R2*Sine(U), Ztarget=fixed]. Referring to FIG. 3, the second dimension R2 of the eye 200 may be obtained as a function of the desired viewing angle 260 (V0) and a dimension of the eye 200, e.g., first dimension R1 (eye radius in the anterior to posterior), as [R2=R1*Sine(V0)]. Referring to FIG. 3, the second dimension R2 of the eye 200 may be based on a third dimension R3, which is the eye radius in the superior to inferior direction. The controller C may be configured to update the coordinate positions of the target point [Xtarget, Ytarget, Ztarget] in the orbital trajectory 230, e.g., by injecting respective delta values in each update cycle. The corrections include anti-yaw correction and roll and pitch amounts for the stereoscopic camera 12.

The corrections (block 410 of FIG. 6) further include automatic focusing corrections. U.S. Provisional Application 63/167,406 (filed Mar. 29, 2021) describes automatic focusing modes for a stereoscopic imaging system and is hereby incorporated by reference in its entirety. The controller C may be programmed with one or more automatic focusing routines, such as first autofocus routine 50 and second autofocus routine 52, shown in FIG. 1. In the first autofocus routine 50, the orbital scanning mode 14 is adapted to maintain focus in an image while the robotic arm 24 is moving the stereoscopic camera 12 and the target site 16 is fixed. In the second autofocus mode 52, the orbital scanning mode 14 is adapted to keep the image in focus as both the robotic arm 24 and the target site 16 move. The location of the target point 148 [Xtarget, Ytarget, Ztarget] is continually adjusted, e.g., by automatically adjusting the focus motor corresponding to any total focal length change.

An example implementation of the first autofocus routine 50 is described below. First, the controller C is programmed to calculate the change in working span (ΔW) relative to a previously saved value (e.g., previous iteration or initialized value) due to the movement of the stereoscopic camera 12 via the robotic arm 24 and obtain an updated value of the working span (W). In other words, displacement of the working span (W) due to robotic movement is tracked in each movement cycle. Second, the controller C is programmed to calculate and transmit motor commands for the focus motor 110 corresponding to the updated value of the working span (W). A calibrated look-up-table can be employed to convert the change in working span to commands for the focus motor 110. The radius of the virtual sphere 300 in each iteration is reset to be the updated value of the working span (W), with internal calculations updating the radius of the virtual sphere 300 each cycle. If the target site 16 is moving, the selected point (at the center 304 of the virtual sphere 300) is replaced with a dynamic trajectory that corresponds to the motion of the target site 16. At the end of all movement of the robotic arm 24, the focus motor 110 is moved to the location of maximum sharpness. The amount of this adjustment (moving to the location of maximum sharpness) may be small, due to continuous tracking during operation of the robotic arm 24. This results in an image that always appears in focus, even if the robotic arm 24 is changing the working span. The allowable change in working span W may be limited such that the radius of the virtual sphere 300 does not exceed the maximum and minimum working span permitted by the hardware. In some embodiments, a feature flag may be inserted to enforce artificial robot boundaries to prevent the robotic arm 24 from exceeding these limitations on the working span W.

An example implementation of the second autofocus routine 52 is described below. The second autofocus routine 52 of FIG. 1 is adapted to keep an image in focus as the robotic arm 24 moves, independent of movement type, and allow for smooth, low-noise maximum sharpness adjustments at the end of the move. This means the image remains in focus, even if the stereoscopic camera 12 is rotating to view different scenes, the scene itself is changing depth, the working span is changing, or all of the above at the same time. The second autofocus routine 52 measures relative changes in focal length and keeps the image in focus based on the deviations from the starting values, even as the robotic arm 24 is moving, and the target location, depth, and working span may be changing. Stated differently, the second autofocus mode 52 requires an initial set of starting values or estimates of the target location (in 3D space) and focal length. The initial set of starting values may be fed into the controller C as an output of a sub-routine or machine-learning algorithm. For example, the starting values may be obtained when a sharpness control routine has been successfully performed once during the application.

First, the controller C is programmed to determine a change in height due to movement of the robotic arm 24 (inputted in block 402). The height is defined as the change in position of the stereoscopic camera 12 along the axial direction (Z axis here) from movement of the robotic arm 24. The controller C may calculate the change in height using position data of the joints (e.g., joint sensor 33 of FIG. 1) and other parts of the robotic arm 24.

Second, the controller C is programmed to determine a change in target depth (ΔZ_target). The controller C may receive input data pertaining to a disparity signal in order to calculate the change in target depth. The change in target depth (ΔZ_target) may be calculated using feedback control with a closed-loop control module, which may be a PI controller, a PD controller and/or a PID controller. In one example, the change in target depth (ΔZ_target) is calculated using a PID controller and disparity values as follows:

ΔZ_target=Kp(Rc−Rt)+Ki∫(Rc−Rt)dt−Kd*dRc/dt

Here, Rc is the current disparity value, and Rt is the initial target disparity value, defined as the disparity value recorded when the starting values were initialized and stored. Here, Kp, Ki and Kd are the proportional, integral and derivative constants, respectively, from the PID controller, with the process variable being a difference between the current disparity value (Rc) and the initial target disparity (Rt). The constants Kp, Ki and Kd may be obtained via calibration with known changes in target depth (changes along Z axis here).

Third, the controller C is programmed to determine a change in location coordinates of the target site 16 based on the change in target depth. The stored location of the Z component of the target site 16 is updated as:

Z_updated=Z_initial−ΔZ_target

Finally, the controller C is programmed to determine a combined focal length change (ΔF) and update the specific focal length (F). At each update cycle, the specific focal length F is updated using a feed-forward term from the robotic arm 24 plus a feedback term from the target depth disparity, as shown below:

ΔF=ΔF_robot+ΔF_target

The specific focal length F is updated by two elements: firstly, by how much the terminating point of the view vector 118 of the stereoscopic camera 12 has changed, and secondly by how much the target depth of the target 16 has changed. Because the two elements are in different frames of reference, a homogenous transformation matrix (4×4) is employed to transform base coordinates (Xbase, Ybase, Zbase) in a robotic base frame to a camera coordinate frame. The base coordinates (Xbase, Ybase, Zbase) represent the instant or current location of the target site 16 in a robotic base frame. The homogenous transformation matrix is composed of a rotation matrix Q (3×3) and translation vector P and may be represented as:

$$[ \begin{matrix}Q & P \\ 0 & 1\end{matrix} ] .$$

The rotation matrix Q has 3 by 3 components, while the translation vector P has 3 by 1 components. The translation vector P represents the offset (x0, y0, z0) from the robotic base frame to the camera frame, where the origin of the robotic base frame is zero (0, 0, 0) and the origin of the camera frame in the robotic base frame is (x0, y0, z0). The equation below converts the base coordinates (in the robotic base frame) to the camera frame.

$$[ \begin{matrix}Xbase \\ Ybase \\ Zbase \\ 1\end{matrix} ] = [ \begin{matrix}Q & p \\ 0 & 1\end{matrix} ] [ \begin{matrix}0 \\ 0 \\ F \\ 1\end{matrix} ] , where P = [ \begin{matrix}x 0 \\ y 0 \\ z 0 \\ 1\end{matrix} ] and Q = [ \begin{matrix}x 1 & x 2 & x 3 \\ y 1 & y 2 & y 3 \\ z 1 & z 2 & z 3\end{matrix} ] . \qquad ( eq . 1 )$$

Expanding this equation results in:

$$[ \begin{matrix}Xbase \\ Ybase \\ Zbase \\ 1\end{matrix} ] = F [ \begin{matrix}x 3 \\ y 3 \\ z 3 \\ 1\end{matrix} ] + [ \begin{matrix}x 0 \\ y 0 \\ z 0 \\ 1\end{matrix} ] \qquad ( eq . 2 )$$

Here the vector (x3, y3, z3) represents the third column of the rotational matrix Q, which is the Z basis vector of the rotational space of the transformation matrix. The z position component of the Z basis vector is represented by z3. The updated value of the specific focal length F may be calculated using the following equations:

$$\begin{matrix}Zbase = F ( z 3 ) + z 0 & ( eq . 3 ) \\ F = ( \frac{Zbase - z 0}{z 3} ) & ( eq . 4 )\end{matrix}$$

Advancing from block 410 to block 412 in FIG. 6, the controller C is configured to send out updated motor commands for performing the orbital trajectory 230. The motor commands corresponding to the updated value of the specific focal length F are calculated and transmitted. The focus motor 110 is moved the correct amount, determined through calibration, such that the working span is the same as the updated value of the specific focal length F. As noted above, the orbital trajectory 230 can be performed by holding the second sphere angle (V) constant at the desired viewing angle 260, while iterating movement along the first sphere angle (U) between a predefined starting angle (Uᵢₙᵢₜᵢₐₗ) and a predefined ending angle (U_{final}). When the robotic arm 24 stops moving at the end of the orbital trajectory 230, the controller C is configured to determine motor commands for the focus motor 110 corresponding to a maximum sharpness position. The maximum sharpness position is based on one or more sharpness parameters, including a sharpness signal, a maximum sharpness signal and a derivative over time of the maximum sharpness. In some embodiments, the sharpness signal is defined as a contrast between respective edges of an object in the stereoscopic image. The maximum sharpness signal is defined as a largest sharpness value observed during a scan period.

The controller C of FIG. 1 may include or otherwise have access to information downloaded from remote sources and/or executable programs. Referring to FIG. 1, the controller C may be configured to communicate with a remote server 60 and/or a cloud unit 62, via a network 64. The remote server 60 may be a private or public source of information maintained by an organization, such as for example, a research institute, a company, a university and/or a hospital. The cloud unit 62 may include one or more servers hosted on the Internet to store, manage, and process data.

The network 64 may be a serial communication bus in the form of a local area network. The local area network may include, but is not limited to, a Controller Area Network (CAN), a Controller Area Network with Flexible Data Rate (CAN-FD), Ethernet, blue tooth, WIFI and other forms of data. The network 64 may be a Wireless Local Area Network (LAN) which links multiple devices using a wireless distribution method, a Wireless Metropolitan Area Network (MAN) which connects several wireless LANs or a Wireless Wide Area Network (WAN) which covers large areas such as neighboring towns and cities. Other types of connections may be employed.

The controller C of FIG. 1 may be an integral portion of, or a separate module operatively connected to the robotic imaging system 10. The controller C includes a computer-readable medium (also referred to as a processor-readable medium), including a non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic media, a CD-ROM, DVD, other optical media, punch cards, paper tape, other physical media with patterns of holes, a RAM, a PROM, an EPROM, a FLASH-EEPROM, other memory chips or cartridges, or other media from which a computer can read.

Look-up tables, databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store may be included within a computing device employing a computer operating system such as one of those mentioned above and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL language mentioned above.

The flowcharts presented herein illustrate an architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by specific purpose hardware-based devices that perform the specified functions or acts, or combinations of specific purpose hardware and computer instructions. These computer program instructions may also be stored in a computer-readable medium that can direct a controller or other programmable data processing apparatus to function in a particular manner, such that the instructions stored in the computer-readable medium produce an article of manufacture including instructions to implement the function/act specified in the flowchart and/or block diagram blocks.

The numerical values of parameters (e.g., of quantities or conditions) in this specification, including the appended claims, are to be understood as being modified in each respective instance by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of each value and further divided ranges within the entire range. Each value within a range and the endpoints of a range are hereby disclosed as separate embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A robotic imaging system for imaging a target site in an eye, the robotic imaging system comprising:
a stereoscopic camera configured to record a left image and a right image of the target site for producing at least one stereoscopic image of the target site;
a robotic arm operatively connected to the stereoscopic camera, the robotic arm being adapted to selectively move the stereoscopic camera relative to the target site;
wherein the stereoscopic camera includes an optical assembly having at least one lens and defining a working span, the optical assembly having at least one focus motor adapted to move the at least one lens to selectively vary the working span;
a controller in communication with the robotic arm and having a processor and tangible, non-transitory memory on which instructions are recorded, the controller being configured to determine a change in target depth from an initial target position, the change in the target depth being defined as a displacement in position of the target site along an axial direction;
wherein the controller is configured to update a specific focal length based in part on the change in the target depth; and
wherein the controller is adapted to selectively execute an orbital scanning mode causing the robotic arm to sweep an orbital trajectory at least partially circumferentially around the eye while maintaining focus.

2. The robotic imaging system of claim 1, wherein the target site includes an orra serrata of the eye.

3. The robotic imaging system of claim 1, wherein:
the orbital trajectory is defined in a spherical coordinate axis defining a first spherical angle and a second spherical angle; and
the controller is adapted to change a view angle of the orbital trajectory by keeping the first spherical angle constant while iterating the second spherical angle until a desired viewing angle is reached.

4. The robotic imaging system of claim 3, wherein:
the controller is adapted to selectively command the orbital trajectory by iterating the first spherical angle between a predefined starting angle and a predefined ending angle while keeping the second spherical angle constant at the desired viewing angle.

5. The robotic imaging system of claim 1, wherein the orbital trajectory at least partially forms a circle.

6. The robotic imaging system of claim 1, wherein the orbital trajectory at least partially forms an ellipsoid.

7. The robotic imaging system of claim 1, wherein the orbital trajectory subtends an angle between about 180 degrees and 300 degrees.

8. The robotic imaging system of claim 1, wherein the orbital trajectory subtends an angle of about 360 degrees.

9. The robotic imaging system of claim 1, wherein:
the controller is configured to center the stereoscopic camera on a reference plane of the eye and estimate a first working span to a reference surface of the eye; and
the controller is adapted change a view vector of the stereoscopic camera to a desired viewing angle.

10. The robotic imaging system of claim 1, wherein:
the controller is configured to lock a respective position of each target point along the orbital trajectory by restricting the respective position of the stereoscopic camera to an outer surface of a virtual sphere, the virtual sphere defining a radius equal to the specific focal length.

11. The robotic imaging system of claim 1, wherein:
the specific focal length is based in part on a desired viewing angle, a dimension of the eye and a first working span.

12. The robotic imaging system of claim 10, wherein:
the controller is configured to determine a change in height of the stereoscopic camera from an initial camera position, the change in the height being defined as a displacement in position of the stereoscopic camera along an axial direction; and
the controller is configured to update the specific focal length based in part on the change in the height of the stereoscopic camera.

13. The robotic imaging system of claim 1, wherein:
when the robotic arm is no longer moving, the controller is configured to determine motor commands for the at least one focus motor corresponding to a maximum sharpness position; and
wherein the maximum sharpness position is based on one or more sharpness parameters, including a sharpness signal, a maximum sharpness signal and a derivative over time of the maximum sharpness.

14. The robotic imaging system of claim 1, wherein:
in each update cycle, the controller is configured to inject respective delta values to respective coordinate positions of the orbital trajectory.

15. A stereoscopic imaging system for imaging a target site in an eye, the stereoscopic imaging system comprising:
a stereoscopic camera configured to record a left image and a right image of the target site for producing at least one stereoscopic image of the target site;
a robotic arm operatively connected to the stereoscopic camera, the robotic arm being adapted to selectively move the stereoscopic camera relative to the target site;
wherein the stereoscopic camera includes an optical assembly having at least one lens and defining a working span, the optical assembly having at least one focus motor adapted to move the at least one lens to selectively vary the working span;
a controller in communication with the robotic arm and having a processor and tangible, non-transitory memory on which instructions are recorded; and
wherein the controller is adapted to selectively execute an orbital scanning mode causing the robotic arm to sweep an orbital trajectory at least partially circumferentially around the eye while maintaining focus.

16. The stereoscopic imaging system of claim 15, wherein the target site includes an orra serrata of the eye.

17. The stereoscopic imaging system of claim 15, wherein:
the orbital trajectory is defined in a spherical coordinate axis defining a first spherical angle and a second spherical angle; and
the controller is adapted to change a view angle of the orbital trajectory by keeping the first spherical angle constant while iterating the second spherical angle until a desired viewing angle is reached; and
the controller is adapted to selectively command the orbital trajectory by iterating the first spherical angle between a predefined starting angle and a predefined ending angle while keeping the second spherical angle constant at the desired viewing angle.

18. The stereoscopic imaging system of claim 15, wherein:
when the robotic arm is no longer moving, the controller is configured to determine motor commands for the at least one focus motor corresponding to a maximum sharpness position; and
wherein the maximum sharpness position is based on one or more sharpness parameters, including a sharpness signal, a maximum sharpness signal and a derivative over time of the maximum sharpness.

19. The stereoscopic imaging system of claim 18, wherein:
the sharpness signal is defined as a contrast between respective edges of an object in the at least one stereoscopic image; and
the maximum sharpness signal is defined as a largest sharpness value observed during a scan period.
